(19) United States
(12) Patent Application Publication
(10) Pub. No.: US 2024/0021303 A1
(43) Pub. Date: Jan. 18, 2024

(54) APPARATUS, METHOD AND COMPUTER PROGRAM FOR TREATING BENIGN PAROXYSMAL POSITIONAL VERTIGO

(71) Applicant: Imperial College Innovations Limited, London (GB)

(72) Inventors: Barry SEEMUNGAL, London (GB); Danilo MANDIC, London (GB)

(73) Assignee: Imperial College Innovations Limited, London (GB)

(21) Appl. No.: 18/036,753

(22) PCT Filed: Nov. 12, 2021
(86) PCT No.: PCT/GB2021/052935

§ 371 Date: May 12, 2023

(30) Foreign Application Priority Data
Nov. 13, 2020 (GB) 2017933.9

(51) Int. Cl. G16H 40/63; G06F 1/16; G06F 3/0346

(52) U.S. Cl. CPC G16H 40/63; G06F 1/163; G06F 3/0346

(57) ABSTRACT

An apparatus for treating benign paroxysmal positional vertigo, BPPV, comprises at least a processing unit and a feedback mechanism. The processing unit is configured to receive data indicative of a manoeuvre carried out by a user and to compare the received data to data indicative of a predefined manoeuvre for treating BPPV, and the feedback mechanism is configured to provide feedback to the user in accordance with the result of the comparison between the received data and the predefined manoeuvre. In some embodiments the feedback may be provided as audio output, for example in the form of intelligible speech descriptive of a similarity and/or a difference between the manoeuvre carried out by the user and the predefined manoeuvre. A corresponding method and computer program for performing the method are also disclosed.

### TECHNICAL FIELD

The present invention relates to the treatment of Benign Paroxysmal Positional Vertigo (BPPV). More particularly, the present invention relates to an apparatus, method and computer program for treatment of BPPV.

### BACKGROUND

Benign paroxysmal positional vertigo (BPPV) is the most common cause of vertigo throughout the world and is believed to affect 0.5% of the population annually. Most patients develop BPPV spontaneously and without an obvious trigger (i.e. idiopathic). The mechanism causing the symptoms and signs of BPPV relates to calcium carbonate crystals that become dislodged from the macula within the utricle, and these crystals, with or without adherent proteinaceous material, become lodged in the semicircular canals (or on the cupula, which is the motion detector mechanism of the canal) in the inner ear. The crystals are denser than the endolymph fluid in the semicircular canals (2.7 g/mL vs 1 g/mL) so the crystals move (or sink) in the canals during head movements that change the orientation of the canals with respect to gravity. The gravity-related movement of the crystals results in the abnormal activation of the ampullary nerve. A person suffering from BPPV may experience symptoms such as feeling that they are spinning around and/or seeing the room spin around, usually for roughly 10 seconds, and is typically triggered by head movement with respect to gravity, e.g. upon turning over in bed. The BPPV condition can also cause falls leading to trauma, such as a broken hip or brain injury. Conversely, BPPV can be caused by head trauma, and hence BPPV is very common in patients suffering from concussion and traumatic brain injury (TBI). Recent work shows that patients with acute traumatic brain injury may have BPPV without vertigo due to the disrupted brain mechanisms of perceiving vertigo. In these cases, BPPV is diagnosed by the observation of a typical eye movement response (‘nystagmus’) on moving the patient's head in a specific orientation with respect to gravity. The loss or attenuation of vertigo perception is also found in other brain diseases such as dementia and in many elderly patients at risk of falls. It is still important to treat BPPV in this situation as BPPV can still cause malaise, nausea and vomiting and most importantly falls, indicating that the treatment of BPPV in patients who do not complain of vertigo (or attenuated or altered sensations of vertigo) is important to reduce risk of falling. BPPV can also occur following a viral inflammation of the inner ear (labyrinthitis), or following ischaemia to the inner ear mechanism due to the blockage of the blood supply to the inner ear mechanism. BPPV can be easily treated with a manoeuvre during which the crystals are maneuvered out of the canals and into the lymph drainage. The manoeuvre requires the patient to move their head in a certain way so as to cause the crystals to become dislodged from the canals. If the manoeuvre is incorrectly performed, then there may be no therapeutic benefit. The manoeuvre is completely harmless and can be performed by the affected individuals themselves.

The invention is made in this context.

### SUMMARY OF THE INVENTION

According to a first aspect of the present invention, there is provided an apparatus for treating benign paroxysmal positional vertigo, BPPV, the apparatus comprising: a processing unit configured to receive data indicative of a manoeuvre carried out by a user and to compare the received data to data indicative of a predefined manoeuvre for treating BPPV; and a feedback mechanism configured to provide feedback to the user in accordance with the result of the comparison between the received data and the predefined manoeuvre.

In some embodiments according to the first aspect, the feedback is indicative of whether the user has successfully carried out the predefined manoeuvre.

In some embodiments according to the first aspect, the feedback includes recommended changes that the user can make relative to the previously-performed manoeuvre as indicated by the received data so as to carry out the predefined manoeuvre correctly.

In some embodiments according to the first aspect, the data indicative of the manoeuvre carried out by the user comprises data captured by a sensor configured to detect movement of the user's head.

In some embodiments according to the first aspect, the sensor comprises a motion sensor in the form of an accelerometer and/or gyroscope.

In some embodiments according to the first aspect, the apparatus comprises the sensor configured to detect the movement of the user's head.

In some embodiments according to the first aspect, the apparatus comprises a housing containing the sensor and configured to be worn in close proximity to an inner ear of the user, such that the data captured by the sensor is representative of movement of the inner ear in three dimensions as the manoeuvre is carried out by the user.

In some embodiments according to the first aspect, the housing is configured to be worn in the external auditory canal, or in or on the auricle.

In some embodiments according to the first aspect, the housing is configured to be worn behind the ear.

In some embodiments according to the first aspect, the housing further contains the processing unit and the feedback mechanism.

In some embodiments according to the first aspect, the housing further contains a communication unit configured to communicate with an external device.

In some embodiments according to the first aspect, the processing unit and the sensor are embodied in physically separate devices, the apparatus comprising a communication unit configured to communicate with the sensor, the processing unit being configured to receive the data from the sensor via the communication unit.

In some embodiments according to the first aspect, the apparatus comprises a video processor configured to process video of the user carrying out the manoeuvre to obtain the data indicative of the manoeuvre carried out by the user.

In some embodiments according to the first aspect, the apparatus comprises an image sensor configured to capture the video of the user carrying out the manoeuvre.

In some embodiments according to the first aspect, the processing unit is configured to obtain orientation data indicative of an orientation of the image sensor at the time of capturing the video of the user carrying out of the manoeuvre, and is configured to take into account the orientation of the image sensor to determine whether the user has successfully carried out the predefined manoeuvre. The orientation data may, for example, be obtained from a suitable sensor arranged so as to detect movements of the image sensor, such as an accelerometer.

In some embodiments according to the first aspect, the apparatus comprises a user authentication unit for authenticating the user, wherein the apparatus is configured to only provide the feedback in response to the user being successfully authenticated by the user authentication unit.

In some embodiments according to the first aspect, the user authentication unit is configured to obtain biometric data relating to the user and to authenticate the user on the basis of the obtained biometric data.

In some embodiments according to the first aspect, the user authentication unit is configured to receive an activation code inputted by the user and to authenticate the user on the basis of the received activation code.

In some embodiments according to the first aspect, the feedback mechanism is configured to provide the feedback as audio output reproduced via one or more speakers.

In some embodiments according to the first aspect, the audio output is in the form of intelligible speech descriptive of a similarity and/or a difference between the manoeuvre carried out by the user and the predefined manoeuvre.

In some embodiments according to the first aspect, the feedback mechanism is configured to provide the feedback as visual output via a graphical user interface.

In some embodiments according to the first aspect, the feedback mechanism is configured to provide the feedback as visual output via a virtual reality device.

In some embodiments according to the first aspect, the apparatus is configured to receive user input indicative of symptoms experienced by the user, determine whether the symptoms are consistent with BPPV, and to only be operable to provide the feedback in dependence on a determination that the symptoms are consistent with BPPV.

In some embodiments according to the first aspect, the processing unit is configured to record whether the user has successfully performed the predefined manoeuvre based on the result of the comparison, and to check whether a number of times that the user has successfully performed the predefined manoeuvre is greater than a threshold, wherein in response to a determination that the user has successfully performed the predefined manoeuvre more than the threshold number of times, the feedback mechanism is configured to output a notification to the user.

According to a second aspect of the present invention, there is provided a method for treating benign paroxysmal positional vertigo, BPPV, the method comprising: receiving data indicative of a manoeuvre carried out by a user; comparing the received data to data indicative of a predefined manoeuvre for treating BPPV; and providing feedback to the user in accordance with the result of the comparison between the received data and the predefined manoeuvre.

According to a third aspect of the present invention, there is provided a non-transitory computer readable storage medium arranged to store computer program instructions which, when executed, perform the method according to the second aspect.

### BRIEF DESCRIPTION OF THE DRAWINGS

Embodiments of the present invention will now be described, by way of example only, with reference to the accompanying drawings, in which:

FIG. 1 illustrates an apparatus for treating BPPV, according to an embodiment of the present invention;

FIG. 2 is a flowchart showing a method of treating BPPV, according to an embodiment of the present invention;

FIG. 3 is a flowchart showing a method for checking whether a user is likely to be suffering from BPPV, according to an embodiment of the present invention;

FIG. 4 illustrates an apparatus for treating BPPV, according to an embodiment of the present invention; and

FIG. 5 is a flowchart showing a method of determining when a user's symptoms may be due to a condition other than BPPV, according to an embodiment of the present invention.

### DETAILED DESCRIPTION

In the following detailed description, only certain exemplary embodiments of the present invention have been shown and described, simply by way of illustration. As those skilled in the art would realise, the described embodiments may be modified in various different ways, all without departing from the scope of the present invention. Accordingly, the drawings and description are to be regarded as illustrative in nature and not restrictive. Like reference numerals designate like elements throughout the specification.

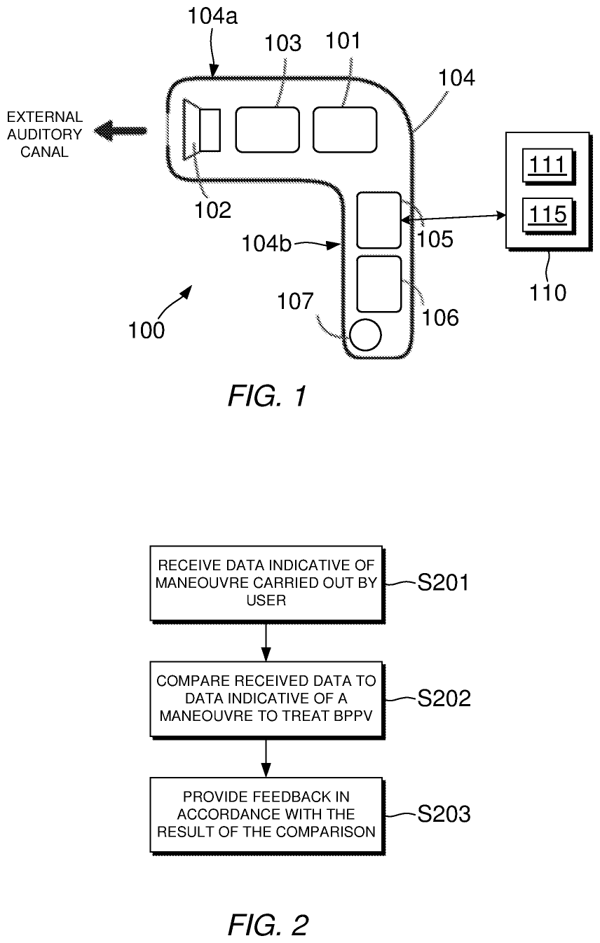

Referring now to FIG. 1, an apparatus for treating BPPV is illustrated according to an embodiment of the present invention. The apparatus 100 comprises a processing unit 101 and a feedback mechanism 102. The feedback mechanism 102 can be any mechanism suitable for providing feedback to a user, and in the present embodiment the feedback mechanism comprises a speaker 102 for providing audio output. In other embodiments different forms of feedback may be provided, including but not limited to visual feedback and tactile feedback, and the feedback mechanism 102 may be adapted accordingly.

Continuing with reference to FIG. 1, the processing unit 101 is configured to receive data indicative of a manoeuvre carried out by the user, and to compare the received data to data indicative of a predefined manoeuvre for treating BPPV. Then, the feedback mechanism is configured to provide feedback to the user in accordance with the result of the comparison between the received data and the predefined manoeuvre. In the present embodiment the feedback is indicative of whether the user has successfully carried out the predefined manoeuvre, meaning that the feedback signals whether or not the manoeuvre was successfully performed. For example, the feedback may comprise a simple yes/no notification to signal whether or not the manoeuvre was successfully performed. In some embodiments the feedback may not include a direct indication as to whether the manoeuvre has been successfully performed, but may instead be representative of any discrepancies between the actual manoeuvre performed by the user and the ‘correct’ manoeuvre, i.e. the predefined manoeuvre for treating BPPV. For example, the feedback may be provided via a display, such as a mobile phone display, tablet, desktop computer screen, or a virtual reality device, and may comprise a visualisation of the actual manoeuvre performed by the user overlain on a visual representation of the correct manoeuvre. It may then be left to the user, and/or another individual such as a healthcare professional, to decide based on the feedback whether the manoeuvre was performed correctly.

For example, in the case of audio output as in the present embodiment, the audio output may take the form of intelligible speech that is descriptive of a similarity and/or a difference between the manoeuvre carried out by the user and the predefined manoeuvre. Depending on the embodiment the audio output may comprise recordings of actual speech, or may comprise synthesised speech.

In some embodiments the feedback may include recommended changes that the user can make relative to the previously-performed manoeuvre so as to carry out the manoeuvre correctly. Here, the previously-performed manoeuvre refers to the user's most recent attempt at performing the manoeuvre, as detected by the sensor 103. For example, the feedback may indicate to the user that they should move their head by a greater or lesser distance in a certain direction during a certain stage in the manoeuvre, and/or may indicate to the user that they should move their head more quickly or more slowly during a certain stage in the manoeuvre. Such recommendations may assist the user in carrying out the manoeuvre correctly.

In some embodiments, the predefined manoeuvre for treating BPPV may be selected from among a plurality of predefined manoeuvres, each of which is designed to treat a different type of BPPV condition. For example, the user may be diagnosed by a suitably trained person such as clinician or therapist, who can examine the patient and find the clinical signs of BPPV based upon the evoked eye movement response (referred to as nystagmus). The clinician may be able to diagnose the side (left or right) and canal (posterior, horizontal or anterior) with high sensitivity/specificity. There are 3 canals that can be affected by BPPV, with a frequency of occurrence as follows: posterior 90%; lateral 9%; anterior 1%. The apparatus can then be programmed to treat the diagnosed BPPV condition by selecting the appropriate manoeuvre from the plurality of predefined manoeuvres, so as to treat the specific canal and side for which the BPPV has been diagnosed. Such embodiments may be particularly advantageous in scenarios in which a patient is diagnosed by a clinician but then has to carry out the treatment at home rather than in the presence of the clinician. For example, this could apply in cases of recurrent BPPV, or in situations where the treatment may provoke unsteadiness or malaise which could may it difficult for the patient to travel home after treatment, in which case it may be preferable to wait until the patient returns home before beginning treatment.

In some embodiments in which the predefined manoeuvre for treating BPPV is selected from among a plurality of predefined manoeuvres, it may be possible for a user to select the manoeuvre themselves without an expert diagnosis, for example by selecting the manoeuvre for treating for treating the side (left or right) that triggers the symptoms on turning over. It may not be necessary to diagnose the canal, since the manoeuvre for treating posterior canal BPPV may be selected by default given that this is the case in 90% of cases of BPPV.

The data indicative of the manoeuvre carried out by the user, which is used by the processing unit 101 to determine whether or not the user has successfully performed the manoeuvre for treating BPPV, can be obtained in different ways depending on the embodiment. For example, the data may be obtained from a sensor that directly detects movement of the user's head. In the present embodiment, the apparatus 100 further comprises a motion sensor 103 configured to detect movement of the user's head. The sensor 103 may comprising a multi-axis inertial device such as an accelerometer and/or gyroscope, which can accurately measure movements of the user's head in three-dimensional space. Data captured by the sensor 103 can be used by the processing unit 101 to check whether the user has correctly performed the manoeuvre.

In embodiments in which a sensor is used to detect movements of the user's head, the apparatus may be configured such that the sensor 103 can be positioned in close proximity to the user's inner ear. In this way, the data captured by the sensor 103 can be more closely representative of the actual movement of the inner ear and the semicircular canal(s) in three dimensions as the manoeuvre is carried out by the user, as opposed to embodiments in which the sensor 103 is positioned further away from the ear. Here, the term ‘close proximity’ is used to denote a location that is sufficiently close to the ear that movements detected by the sensor can be considered to be a sufficiently accurate measure of the actual movement of the user's inner ear to enable a decision to be taken as to whether or not the manoeuvre has been performed correctly. For example, in some embodiments the apparatus may be configured so as to be worn in the ear canal, such that the apparatus will maintain a fixed position relative to the user's inner ear while the user performs the manoeuvre.

Nevertheless, in other embodiments the sensor 103 may be positioned further away from the ear. For example, the sensor 103 may be positioned elsewhere on the user's head at a known location relative to the ear, such as the forehead. In such embodiments the apparatus 100 may have any suitable form to allow the apparatus 100 (and hence the sensor 103) to be attached to the head at a fixed position, including but not limited to: goggles; eye-piece; spectacles; hat; or headband. In some embodiments the apparatus 100 may comprise suitable attachment means for attaching the apparatus 100 to part of the user's body, for example the skin or hair. Examples of suitable attachment means include, but are not limited to: adhesive; low pressure suction cup; mechanical grip mechanism. As yet a further alternative, in some embodiments the apparatus 100 may be fixed subcutaneously, for instance via subcutaneous injection or via surgical implantation. For example, in some embodiments the apparatus 100 may be embodied as a programmable microelectromechanical (MEM) package that includes one or more suitable sensors, such as inertial sensors, and which may be installed with any intracranial implant.

In embodiments in which the sensor 103 is not positioned in close proximity to the ear, if the data indicative of the predefined manoeuvre relates to an expected movement path of the ear when the manoeuvre is performed correctly, the processing unit 101 may determine the movement of the user's inner ear from the movement detected by the sensor, taking into account the relative positions of the sensor 103 and the ear in three-dimensional space. In other words, the processing unit 101 may convert the sensor data into data indicative of the movement of the user's ear, even when the sensor 103 is not positioned in close proximity to the ear. The sensor data can be converted into data indicative of the movement of the user's ear, provided that the locus of movement of a stable anchor point on the head is known (i.e. the position of sensor 103) that will allow the trajectory of the head in space over time to be calculated with respect to gravity. Then, the processing unit 101 may compare the determined movement of the ear to the data indicative of the predefined manoeuvre, to check whether the manoeuvre has been correctly performed. Alternatively, the data indicative of the predefined manoeuvre may relate to the expected movement path of the part of the user's head where the sensor 103 is located, such that the data acquired by the sensor 103 can be compared directly to the data indicative of the predefined manoeuvre without any such conversion step.

In the present embodiment, the apparatus 100 comprises a housing 104 which contains at least the sensor 103 and is configured to be worn in the external auditory canal, which may also be referred to as the externa auditory meatus. In another embodiment the housing 104 may be configured to be worn in or on the auricle, which may commonly be referred to as the ‘ear’ or ‘earlobe’. For example, the housing 104 may be configured to be worn behind the ear. In some embodiments the housing 104 may comprise a securing mechanism to assist in securing the housing 104 in place in or on the earlobe, such as a squeezable balloon, rubber or foam component that can conform to the shape of the user's external auditory canal, and this includes low temperature thermoplastic inserts made conformable to the external auditory canal by an integrated resistive electrical circuit that warms the insert to a safe, above-body temperature (on insertion and removal). Such securing mechanisms may have the advantage of securely fixing the housing 104 in place relative to the external auditory canal or the auricle, such that data captured by the sensor 103 accurately reflects movements of the external auditory canal or the auricle in three-dimensional space.

In the present embodiment the housing 104 further contains the processing unit 101 and the feedback mechanism 102. In this way the apparatus 100 can function as a self-contained standalone device capable of monitoring movements of the user's ear (e.g. by monitoring movements of the user's head, from which movement of head-fixed structures such as the ear and/or inner ear labyrinth can be determined), determining whether the user has correctly performed a manoeuvre for treating BPPV, and providing feedback to the user. In other embodiments, other components of the apparatus too such as the processing unit tot and/or the feedback mechanism 102 may be embodied in a physically separate device to the housing 104 and the sensor 103, and may communicate with the sensor 103 via any suitable wired or wireless interface.

In the present embodiment the apparatus too further comprises a communication unit 105 configured to communicate with another device 110 that is physically separate from the housing 104, such as a smartphone, wearable electronic device, or tablet computer. The other device 110 may comprise its own communication unit 115 capable of communicating with the communication unit 105 of the apparatus too, and may comprise its own processing unit 111 capable of carrying out processing tasks.

In the present embodiment, the housing 104 is approximately ‘L-shaped’ in cross-section, comprising a first part 104a configured to extend partially into the external auditory canal, and a second part 104b coupled to the first part 104a, the second part 104b being configured to sit outside the external auditory canal in use. The first part 104a may comprise a proximal end and a distal end, where the proximal end refers to an end of the first part 104a that sits closest to the tympanic membrane, also referred to as the eardrum, when the apparatus too is in use. The distal end refers to the end of the first part 104b that protrudes from the external auditory canal in use. The second part 104b may be coupled to the distal end of the first part 104a, as in the embodiment shown in FIG. 1.

The speaker 102 may be disposed at or close to the proximal end of the first part 104a, so as to minimise the distance between the speaker 102 and the tympanic membrane. This allows the audio output to be provided at a lower volume level whilst still being audible to the user, compared to if the speaker 102 were located further from the tympanic membrane, in turn helping to conserve power since the speaker 102 can be driven at a lower voltage. The sensor 103 may be located in the first part 104a of the housing 104, such that the sensor 103 is located within the external auditory canal while the device is in use, and therefore can directly measure the movement of the external auditory canal while the user is attempting to perform the manoeuvre. In some embodiments, instead of using a speaker the apparatus too may be configured to provide audio output via bone conduction.

The communication unit 105 may be located in the second part 104b of the housing 104 such that the communication unit 105 sits externally to the user's body when the apparatus 100 is in use, helping to reduce attenuation of wireless signals transmitted between the communication unit 105 in the housing 104 and the communication unit 115 in the other device 110. The power source 106 may be located in the second part 104b of the housing 104, which enables a larger power source 106 to be incorporated since the dimensions of the second part 104b are not limited by the dimensions of the external auditory canal. The user input unit 107 may be located in the second part 104b of the housing, so as to remain accessible to the user when the apparatus 100 is in use (e.g. in the case of a physical interface such as a touch panel or button control), and/or so as to clearly detect spoken commands (in the case of a voice-controlled interface).

The apparatus 100 may use the communication unit 105 to interact with the other device 110 in various ways. For example, the apparatus 100 may communicate with the other device 110 via the communication unit 105 so as to provide feedback to the user via the other device 110. In such embodiments, the feedback may be provided via the other device 110 instead of providing feedback from the apparatus 100 itself, in which case the feedback mechanism 102 within the housing 104 may be omitted. Alternatively, feedback may be provided both via the feedback mechanism 102 within the housing 104 and via the other device 110, for example by providing audio feedback through the speaker 102 and providing complimentary visual feedback through a display screen in the other device no. In some embodiments, visual feedback comprising a simulation of the movement carried out by the user, and/or an indication as to whether the manoeuvre was performed correctly, is displayed on the other device 110. In some embodiments, the comparison between the sensor data and the data indicative of the predefined manoeuvre may be carried out by the processing unit 111 in the other device 110, which may receive the sensor data via the communication unit 115.

It will be appreciated that the apparatus 100 may also comprise other components in addition to those described above. For example, the apparatus 100 may comprise a battery 106 or other suitable power source, and/or a user input unit 107 for receiving user input. Examples of suitable forms of user input unit 107 include, but are not limited to, a microphone, touch-sensitive control, buttons, switches, and dials. In the present embodiment the user input unit 107 comprises a microphone. In some embodiments the processing unit 101 can be configured to interpret verbal commands detected via the microphone 107 so as to implement a voice-controlled user interface.

Referring now to FIG. 2, a flowchart showing a method of treating BPPV is illustrated according to an embodiment of the present invention. The method can be performed by apparatus 100 such as that described above with reference to FIG. 1. To assist in understanding of the invention, the method steps illustrated in FIG. 2 will be described with reference to the embodiment of FIG. 1. However, this should not be construed as meaning that the method of FIG. 2 is restricted to use solely with the embodiment of FIG. 1, and in other embodiments the method shown in FIG. 2 may be performed by apparatus which differs from that shown in FIG. 1.

First, in step S201 the processing unit 101 receives data indicative of a manoeuvre carried out by a user, from the sensor 103. Next, in step S202 the processing unit 101 compares the received data to data indicative of a predefined manoeuvre for treating BPPV. The data indicative of the predefined manoeuvre may be stored in internal memory within the apparatus, or may be retrieved from external storage, for example from the other device 110 or from a server. Then, in step S203 feedback is provided to the user via the feedback mechanism 102, in accordance with the result of the comparison between the received data and the predefined manoeuvre. In this way the apparatus can automatically monitor the user's movements and assist them in understanding whether the manoeuvre has been performed correctly, without requiring external assistance.

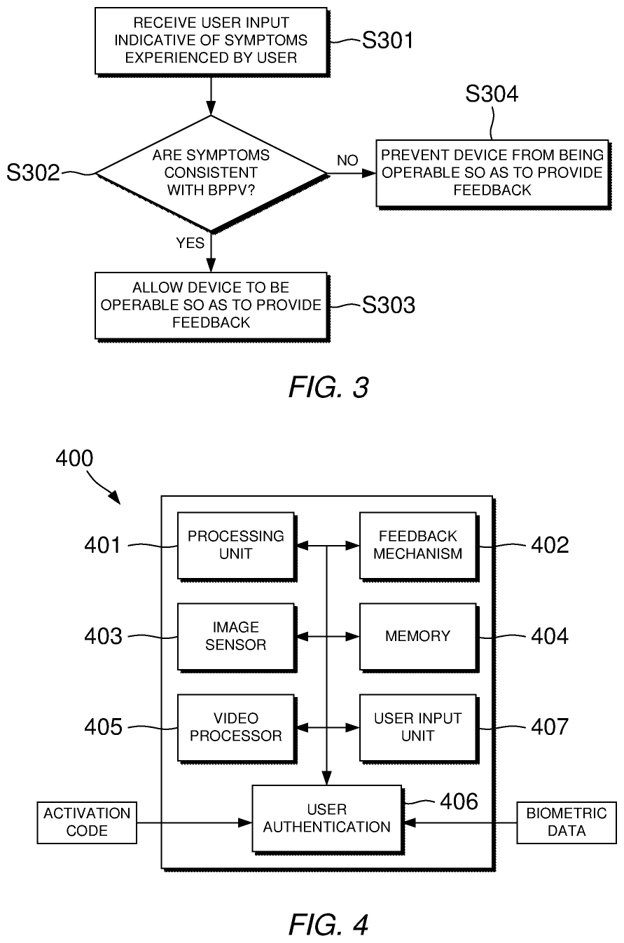

Referring now to FIG. 3, a flowchart showing a method for checking whether a user is likely to be suffering from BPPV is illustrated, according to an embodiment of the present invention. As with FIG. 2, the method of FIG. 3 will be described with reference to the embodiment of FIG. 1 so as to assist in understanding of the invention, but this should not be construed as meaning that the method of FIG. 3 is restricted to use solely with the embodiment of FIG. 1. In an embodiment such as the one shown in FIG. 1, the method steps illustrated in FIG. 3 may either be performed by the processing unit 101 included in the same housing 104 as the sensor 103, or by the processing unit 111 included in the other device 110. In some embodiments, one or more of the steps may be performed by the processing unit 101 included in the same housing 104 as the sensor 103, whilst one or more other steps in the method may be performed by the processing unit 111 included in the other device 110.

First, in step S301 the processing unit 101, 111 receives user input indicative of symptoms experienced by the user. For example, such user input may be received via a user input unit 107 contained within the same physical device as the processing unit 101, or may be received via another device 110. Next, in step S302 the processing unit 101, 111 checks whether the symptoms reported by the user in the user input are consistent with BPPV. An example of a symptom that is consistent with BPPV is that the vertigo is triggered by lying back and/or turning over in bed. If the user input indicates that this is the case, then the symptom is deemed to be consistent with BPPV, and the processing unit 101, 111 proceeds to step S303. In step S303, the apparatus is permitted to be operable so as to monitor the user's movements and provide feedback, as described above with reference to FIGS. 1 and 2.

Alternatively, if the user input indicates that the vertigo is triggered by a different movement, such as standing upright, the processing unit 101, 111 proceeds to step S304, and prevents the apparatus from being operable to provide feedback. In this way, it can be ensured that the apparatus is only used by a user who is likely to be suffering from BPPV. For instance, if the user input indicates that the vertigo is triggered exclusively by standing upright and never when lying back and turning over in bed, then the more likely diagnosis may be postural hypotension rather than BPPV. In some embodiments, the processing unit 101, 111 may control the feedback mechanism 102 to output a notification advising the user of the most likely diagnosis, based on the reported symptoms as indicated in the user input.

Referring now to FIG. 4, an apparatus for treating BPPV is illustrated according to an embodiment of the present invention. The apparatus 400 may perform any of the methods described above with reference to FIGS. 1 to 3. Like the embodiment of FIG. 1, the apparatus 400 comprises a processing unit 401, feedback mechanism 402, and user input unit 407, and for the sake of brevity a detailed description of similar aspects to both embodiments will not be repeated here.

Continuing with reference to FIG. 4, the apparatus 400 comprises an image sensor 403, memory 404, video processor 405, and user authentication unit 406. In other embodiments an apparatus may not comprise all of the components illustrated in FIG. 4, and it will be appreciated that certain ones of the components may be omitted depending on the required functionality. The memory 404 may be embodied as any suitable form of computer-readable storage medium, and may for example be used to store computer program instructions that can be executed by the processing unit 401 so as to perform any of the method described herein. Similarly, such memory may also be incorporated in an embodiment such as the one shown in FIG. 1.

In the present embodiment, the image sensor 403 is configured to capture video of the user carrying out the manoeuvre, and the video processor 405 is configured to process the video to obtain the data indicative of the manoeuvre carried out by the user. Depending on the embodiment, the image sensor 403 may capture still images and/or video at visible wavelengths and/or at other wavelengths of electromagnetic radiation, for example infrared. For example, the video processor 405 may execute an image processing algorithm to automatically detect a position of the user's head in a plurality of frames of the captured video, and to track movements of the user's head during the video. The video processor 405 and/or the processing unit 401 may also take into account data from one or more other sensors which is indicative of the direction of gravity in the captured video, for example an accelerometer or gyroscope whose orientation relative to the image sensor 403 is known, so as to determine the position of the user's head relative to the direction of gravity. In the present embodiment the image sensor 403 and the video processor 405 are embodied in the same physical device, but in other embodiments the video processor 405 and the image sensor 403 may be embodied in physically separate devices. In some embodiments, the video processor 405 may be embodied as software instructions executed on the processing unit 401.

In some embodiments the apparatus may be embodied as a handheld device, such as a smartphone, and may capture video of the user's eye movements from a front-facing camera operating in a so-called ‘selfie’ mode. When combined with data indicative of the orientation and movements of the handheld device, for example obtained from an accelerometer and/or gyroscope included in the handheld device, together with data indicative of the orientation and movements of the user's head (from the device worn on or near the inner ear) can be used to diagnose the canal involved, and/or whether the triggered nystagmus is likely to indicate a brain problem. Measurement of nystagmus by a handheld device can be useful during the procedure in some patients (e.g. TBI, dementia or elderly patients) who do not complain of vertigo.

The user authentication unit 406 is configured to authenticate the user, and the apparatus is configured to only provide the feedback in response to the user being successfully authenticated by the user authentication unit 406. Depending on the embodiment, different forms of authentication may be used. In some embodiments, the user authentication unit 406 may be configured to receive an activation code inputted by the user and to authenticate the user on the basis of the received activation code. For example, an authentication code may be printed on packaging of the apparatus 400, or may be downloaded in the case of online activation. In some embodiments the activation code may be provided by a healthcare professional, for example in the form of a prescription, such that the apparatus may only be used in circumstances when it has been prescribed as a form of treatment.

In another embodiment, the user authentication unit 406 may be configured to obtain biometric data relating to the user and to authenticate the user on the basis of the obtained biometric data. For example, when a user authentication unit 406 is included in an embodiment such as the one shown in FIG. 1, the other device (e.g. a smartphone 110) may be used to play a certain audio output, such as a series of bleeps, whilst the apparatus 100 is situated in the ear. The bleeps can then be recorded via the microphone 107. The signal recorded by the microphone 107 will be unique to that particular individual, since the ear morphometry will filter the sounds in a way that is specific for the individual. In this way, the signal recorded by the microphone 107, or data that is extracted from the microphone signal or is otherwise derived from the microphone signal, can be used as biometric data and provided as an input to the user authentication unit 406.

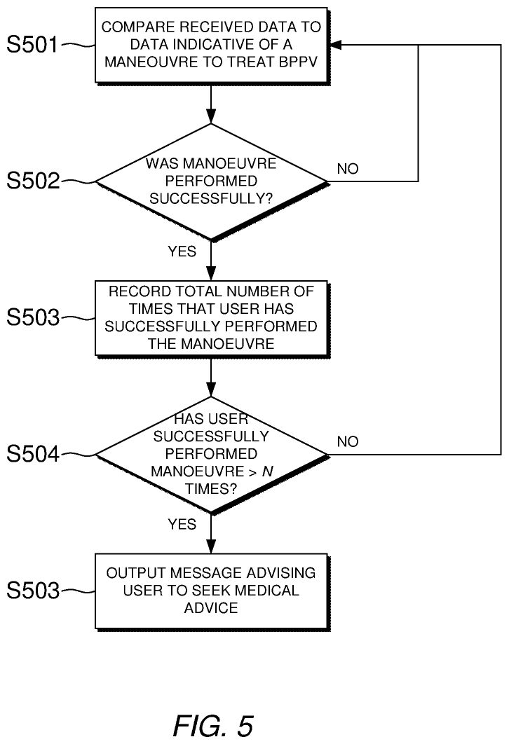

Referring now to FIG. 5, a flowchart showing a method of determining when a user's symptoms may be due to a condition other than BPPV is illustrated, according to an embodiment of the present invention. For convenience the method of FIG. 5 will be described with reference to the apparatus 400 of the embodiment in FIG. 4. However, this should not be construed as limiting, and it should be understood that the method shown in FIG. 5 can be performed by any of the processing units 101, 111, 401 described above in relation to FIGS. 1 and 4, or could be performed by any other embodiment of the apparatus.

First, in step S501 the processing unit 401 compares the received data to the data indicative of the predefined manoeuvre, as described above. Then, in step S502 the processing unit 401 determines based on the result of the comparison whether the predefined manoeuvre was performed successfully. If not, then the processing unit 401 returns to step S501 and waits to receive new data (i.e. for the user to attempt the manoeuvre again). On the other hand, if the predefined manoeuvre was performed successfully, then the processing unit 401 proceeds to step S503 and records the total number of times that the user has successfully performed the predefined manoeuvre. For example, the processing unit 401 may increment a counter stored in the memory 404 each time that it is determined that the user has successfully performed the predefined manoeuvre. Then, in step S504 the processing unit 401 checks whether the number of times that the user has successfully performed the predefined manoeuvre is greater than a threshold number of times, N.

If the threshold has not yet been reached, then the processing unit 401 returns to step S501 and waits to receive new data. On the other hand, in response to a determination in step S504 that the user has successfully performed the predefined manoeuvre more than the threshold number of times, in step S503 the processing unit 401 controls the feedback mechanism 402 to output a notification to the user. The notification may take different forms in different embodiments, and may indicate that the user should seek medical attention. This is because reaching the threshold number of successful attempts may be an indication that the user is suffering from a condition other than BPPV, on the basis that the user is presumably attempting to repeat the manoeuvre because their symptoms have not improved, despite having successfully performed the procedure more than N times. Examples of other conditions that may produce similar symptoms to BPPV but which may not respond to the BPPV repositioning manoeuvre (i.e. the predefined manoeuvre) include, but are not limited to, non-benign brain conditions such as those affecting brainstem structures.

The threshold N may be set based on a number of treatments (i.e. successful manoeuvres) that would typically be expected to result in an improvement in symptoms in a user suffering from BPPV. For example, N may be set to three, such that after the fourth successful attempt at performing the manoeuvre the user is prompted to seek medical advice for an expert diagnosis. By setting a threshold in this way and outputting a notification once the threshold is exceeded, the apparatus can encourage the user to seek prompt medical attention in a situation where they may be suffering from an as-yet undiagnosed condition other than BPPV. It will be appreciated that an entirely equivalent approach would be to set the threshold one higher (e.g. 4 rather than 3), and then to check in step S504 whether the number of successful attempts is equal to the threshold as opposed to exceeding the threshold.

In some embodiments, the apparatus may be configured to instruct the user to perform a diagnostic manoeuvre after every N therapeutic manoeuvres (i.e. once the user has performed the predefined manoeuvre for treating BPPV a total of N times), where N≤1. The diagnostic manoeuvre is a manoeuvre that is designed to trigger BPPV symptoms if the condition still persists, and can therefore be used to verify whether or not the treatment has been successful. The apparatus may check whether the user has correctly performed the diagnostic manoeuvre in a similar manner to that described above for checking whether the user has correctly performed a predefined manoeuvre for treating BPPV, for example by using one or more sensors such as an image sensor and/or accelerometer to obtain data indicative of movement of the user's ear while carrying out the diagnostic manoeuvre.

If the apparatus then determines that the user has correctly performed the diagnostic manoeuvre and there are now no symptoms of BPPV, then the treatment can be considered successful and no further treatment manoeuvres may be required, unless otherwise instructed by a healthcare physician. Some patients with BPPV, especially the elderly, those with dementia or brain injury, may display worse balance from BPPV but may not complain of vertigo. Hence, the device can instruct the user to perform a balance task, during which postural sway can be measured by the device, both before and after the repositioning manoeuvre. Since postural sway improves following BPPV treatment, an additional metric of monitoring response to BPPV treatment is postural sway, as measured by the device. In some embodiments the apparatus may instruct the user to perform a predefined number M of consecutive diagnostic manoeuvres before determining whether or not the treatment can be considered successful, where M≤1. One or both of the numbers of treatment manoeuvres and diagnostic manoeuvres, N and M, may be default values pre-programmed into the apparatus, or may be set by the user and/or by a physician.

In some embodiments, instead of or in addition to outputting the notification at step S503, once the threshold number of successful attempts is exceeded in step S504 the apparatus 400 may be configured to automatically transmit a notification to a healthcare authority or medical professional for expert assessment, who may then independently make contact with the user.

Whilst certain embodiments of the invention have been described herein with reference to the drawings, it will be understood that many variations and modifications will be possible without departing from the scope of the invention as defined in the accompanying claims.

1. An apparatus for treating benign paroxysmal positional vertigo, BPPV, the apparatus comprising:
a processing unit configured to receive data indicative of a manoeuvre carried out by a user and to compare the received data to data indicative of a predefined manoeuvre for treating BPPV; and
a feedback mechanism configured to provide feedback to the user in accordance with the result of the comparison between the received data and the predefined manoeuvre.

2. The apparatus of claim 1, wherein the feedback is indicative of whether the user has successfully carried out the predefined manoeuvre, and/or
wherein the feedback includes recommended changes that the user can make relative to the previously-performed manoeuvre as indicated by the received data so as to carry out the predefined manoeuvre correctly.

3. (canceled)

4. The apparatus of claim 1, 2 or 3, wherein the data indicative of the manoeuvre carried out by the user comprises data captured by a sensor configured to detect movement of the user's head.

5. The apparatus of claim 4, wherein the sensor comprises a motion sensor in the form of an accelerometer and/or gyroscope, and/or
wherein the apparatus comprises the sensor configured to detect the movement of the user's head.

6. (canceled)

7. The apparatus of claim 6, comprising:
a housing containing the sensor and configured to be worn in close proximity to an inner ear of the user, such that the data captured by the sensor is representative of movement of the inner ear in three dimensions as the manoeuvre is carried out by the user.

8. The apparatus of claim 7, wherein the housing is configured to be worn in the external auditory canal, or in or on the auricle.

9. The apparatus of claim 8, wherein the housing is configured to be worn behind the ear.

10. The apparatus of claim 7, 8 or 9, wherein the housing further contains the processing unit and the feedback mechanism.

11. The apparatus of claim 10, wherein the housing further contains a communication unit configured to communicate with an external device.

12. The apparatus of claim 4 or 5, wherein the processing unit and the sensor are embodied in physically separate devices, the apparatus comprising:
a communication unit configured to communicate with the sensor, the processing unit being configured to receive the data from the sensor via the communication unit.

13. The apparatus of claim 1, 2 or 3, comprising:
a video processor configured to process video of the user carrying out the manoeuvre to obtain the data indicative of the manoeuvre carried out by the user.

14. The apparatus of claim 13, comprising:
an image sensor configured to capture the video of the user carrying out the manoeuvre.

15. The apparatus of claim 14 when dependent on claim 2, wherein the processing unit is configured to obtain orientation data indicative of an orientation of the image sensor at the time of capturing the video of the user carrying out of the manoeuvre, and is configured to take into account the orientation of the image sensor to determine whether the user has successfully carried out the predefined manoeuvre.

16. The apparatus of any one of the preceding claims, comprising:
a user authentication unit for authenticating the user, wherein the apparatus is configured to only provide the feedback in response to the user being successfully authenticated by the user authentication unit.

17. The apparatus of claim 16, wherein the user authentication unit is configured to obtain biometric data relating to the user and to authenticate the user on the basis of the obtained biometric data, and/or
wherein the user authentication unit is configured to receive an activation code inputted by the user and to authenticate the user on the basis of the received activation code.

18. (canceled)

19. The apparatus of any of the preceding claims, wherein the feedback mechanism is configured to provide the feedback as visual output via a graphical user interface or a via a virtual reality device, and/or
wherein the feedback mechanism is configured to provide the feedback as audio output reproduced via one or more speakers.

20. The apparatus of claim 19, wherein the audio output is in the form of intelligible speech descriptive of a similarity and/or a difference between the manoeuvre carried out by the user and the predefined manoeuvre.

21. (canceled)

22. The apparatus of any one of the preceding claims, wherein the apparatus is configured to receive user input indicative of symptoms experienced by the user, determine whether the symptoms are consistent with BPPV, and to only be operable to provide the feedback in dependence on a determination that the symptoms are consistent with BPPV, and/or
wherein the processing unit is configured to record whether the user has successfully performed the predefined manoeuvre based on the result of the comparison, and to check whether a number of times that the user has successfully performed the predefined manoeuvre is greater than a threshold, and wherein in response to a determination that the user has successfully performed the predefined manoeuvre more than the threshold number of times, the feedback mechanism is configured to output a notification to the user.

23. (canceled)

24. A method for treating benign paroxysmal positional vertigo, BPPV, the method comprising:
receiving data indicative of a manoeuvre carried out by a user;
comparing the received data to data indicative of a predefined manoeuvre for treating BPPV; and
providing feedback to the user in accordance with the result of the comparison between the received data and the predefined manoeuvre.

25. A non-transitory computer readable storage medium arranged to store computer program instructions which, when executed, perform the method of claim 24.
